(19) United States
(12) Patent Application Publication
(10) Pub. No.: US 2013/0204637 A1
(43) Pub. Date: Aug. 8, 2013

(54) PHARMACY WORKFLOW MANAGEMENT SYSTEM

(71) Applicant: CareFusion 303, Inc., San Diego, CA (US)

(72) Inventors: Timothy W. Vanderveen, Poway, CA (US); Federico Garibaldi, Encinitas, CA (US)

(73) Assignee: CareFusion 303, Inc., San Diego, CA (US)

(21) Appl. No.: 13/802,443

(22) Filed: Mar. 13, 2013

Related U.S. Application Data
(63) Continuation-in-part of application No. 09/860,865, filed on May 18, 2001.
Application No. 09/860,865 is a continuation-in-part of application No. 10/361,704, filed on Feb. 9, 2003.
Application No. 10/361,704 is a continuation-in-part of application No. 10/750,032, filed on Dec. 31, 2003.
Application No. 10/750,032 is a continuation-in-part of application No. 13/246,782, filed on Sep. 27, 2011.
Application No. 13/246,782 is a continuation of application No. 12/947,773, filed on Nov. 16, 2010, now Pat. No. 8,340,792.
Application No. 12/947,773 is a continuation of application No. 10/925,511, filed on Aug. 25, 2004, now Pat. No. 7,860,583.
Application No. 10/925,511 is a continuation-in-part of application No. 11/326,145, filed on Dec. 30, 2005.
(60) Provisional application No. 60/205,125, filed on May 18, 2000.
(60) Provisional application No. 60/652,382, filed on Feb. 11, 2005.

(51) Int. Cl. G06Q 50/22

(52) U.S. Cl. CPC G06Q 50/22; G06Q 10/0633
USPC 705/2

(57) ABSTRACT

A pharmacy workflow management system may include a processor and a memory. The memory may include instructions that, when executed by the processor, cause the processor to receive, from at least one healthcare device, at least one information item pertaining to progress of delivery of a first medication of a first order, where additional medication is required for the first order when the delivery is complete, receive a second order for a second medication from a healthcare system, the second medication being different than the first medication, and schedule a first preparation of the additional medication for the first order and a second preparation of the second medication of the second order such that the additional medication is prepared and available upon completion of the delivery of the first medication, where the scheduling is based at least in part on the at least one information item.

### CROSS-REFERENCE TO RELATED APPLICATIONS

The present application is a continuation-in-part of co-pending U.S. patent application Ser. No. 09/860,865, entitled “Distributed Remote Asset and Medication Management Drug Delivery System,” filed on May 18, 2001, which claims priority to U.S. Provisional Patent Application Ser. No. 60/205,125, entitled “Distributed remote asset and medication management drug delivery system (DRAMMDDS),” filed on May 18, 2000, both of which are hereby incorporated by reference in their entirety for all purposes. The present application is also a continuation-in-part of co-pending U.S. patent application Ser. No. 10/361,704, entitled “Medication Management and Event Logger and Analysis System,” filed on Feb. 9, 2003, which is hereby incorporated by reference in its entirety for all purposes. The present application is also a continuation-in-part of co-pending U.S. patent application Ser. No. 10/750,032, entitled “Centralized Medication Management System,” filed on Dec. 31, 2003, which is hereby incorporated by reference in its entirety for all purposes. The present application is also a continuation-in-part of co-pending U.S. patent application Ser. No. 13/246,782, entitled “System and Method for Dynamically Adjusting Patient Therapy,” filed on Sep. 27, 2011, which is a continuation of U.S. patent application Ser. No. 12/947,773, entitled “System and Method for Dynamically Adjusting Patient Therapy,” filed on Nov. 16, 2010, now issued as U.S. Pat. No. 8,340,792, which is a continuation of U.S. patent application Ser. No. 10/925,511, entitled “System and Method for Dynamically Adjusting Patient Therapy,” filed on Aug. 25, 2004, now issued as U.S. Pat. No. 7,860,583, all of which are hereby incorporated by reference in their entirety for all purposes. The present application is also a continuation-in-part of co-pending U.S. patent application Ser. No. 11/326,145, entitled “Management of Pending Medication Orders,” filed on Dec. 30, 2005, which claims priority to U.S. Provisional Patent Application Ser. No. 60/652,382, entitled “Management of Pending Medication Orders,” filed on Feb. 11, 2005, both of which are hereby incorporated by reference in their entirety for all purposes.

### TECHNICAL FIELD

The present description relates generally to a management system, and more particularly, but not exclusively, to a pharmacy workflow management system.

### BACKGROUND

Healthcare facilities, such as hospitals, may utilize many different user devices, healthcare devices, and/or healthcare systems to facilitate with providing healthcare to patients. For example, a healthcare facility may utilize healthcare systems to facilitate with providing healthcare to patients, such as through physician order entry systems, pharmacy information systems, hospital information systems, etc. The healthcare facility may also utilize healthcare devices to facilitate with providing healthcare to patients, such as infusion devices, dispensing devices, respiratory devices, etc. In addition, the healthcare facility may utilize user devices to facilitate with providing healthcare to patients, such as computing stations that are located throughout the health facility, personal digital assistants (PDAs) that are carried by health care professionals, etc. However, communication barriers between these systems and/or devices may prevent the healthcare facility from providing efficient and effective healthcare to patients.

For example, a pharmacy in a healthcare facility may prepare ordered medications to be administered to patients throughout the healthcare facility. In some instances, an order for a medication may need to be refilled as soon as the medication has been administered to patient. However, the pharmacy may not have any insight into if/when the administration of a medication began, and/or if/when the administration will be complete. Thus, the pharmacy may be unable to efficiently manage its workflow to ensure that new orders for medications, and/or refilled orders for medications, are prepared in a timely fashion. Similarly, a healthcare professional, such as a nurse, may have no insight into when an ordered medication, and/or a refilled medication, will be available to administer to a patient. Thus, the healthcare professional may need to expend time and/or resources to contact the pharmacy and inquire upon the status of one or more ordered medications.

### SUMMARY

The disclosed subject matter relates to a method for managing pharmacy workflow. The method may include receiving a plurality of first orders for a plurality of first medications from a healthcare system. The method may further include receiving, from a plurality of healthcare devices, a plurality of information items pertaining to progresses of deliveries of a plurality of second medications of a plurality of second orders, wherein additional medication is required for each of the plurality of second medications when each of the deliveries is complete. The method may further include scheduling a plurality of preparations of the plurality of first orders for the plurality of first medications and the additional medication such that the plurality of first orders for the plurality of first medications are filled in a timely manner and the additional medication is available upon completion of each of the deliveries of each of the plurality of second medications.

The disclosed subject matter also relates to a non-transitory machine readable medium embodying instructions that, when executed by a machine, cause the machine to perform a method for managing pharmacy workflow. The method may include receiving a plurality of first orders for a plurality of first medications from a healthcare system. The method may further include receiving, from a plurality of healthcare devices, information pertaining to progresses of deliveries of a plurality of second medications of a plurality of second orders, wherein additional medication is required for at least one of the plurality of second medications when the delivery of the at least one of the plurality of second medications is complete. The method may further include determining a schedule for preparing the plurality of first orders for the plurality of first medications and the additional medication based at least in part on the progresses of the deliveries of the plurality of second medications.

The disclosed subject matter also relates to a system. The system may include one or more processors and a memory. The memory may include instructions that, when executed by the one or more processors, cause the one or more processors to: receive a plurality of orders, each order being for administration of one of a plurality of medications to one of a plurality of patients with facilitation from one of a plurality of healthcare devices, receive a plurality of information items each pertaining to a progress of the administration of the one of the plurality of medications to one of the plurality of patients with facilitation from one of the plurality of healthcare devices, and provide a report that indicates the progress of the administration of each of the plurality of medications of each of the plurality of orders.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

Certain features of the subject technology are set forth in the appended claims. However, for purpose of explanation, several embodiments of the subject technology are set forth in the following figures.

FIG. 1 illustrates an example network environment in which a pharmacy workflow management system may be implemented in accordance with one or more embodiments.

FIG. 2 illustrates an example messaging architecture in which a pharmacy workflow management system may be implemented in accordance with one or more embodiments.

FIG. 3 illustrates an alternative example messaging architecture in which a pharmacy workflow management system may be implemented in accordance with one or more embodiments.

FIG. 4 illustrates a flow diagram of an example process for a pharmacy workflow management system in accordance with one or more embodiments.

FIG. 5 illustrates a flow diagram of an example process for a user device in a pharmacy workflow management system in accordance with one or more embodiments.

FIG. 6 illustrates a flow diagram of an example process for a user device in a pharmacy workflow management system in accordance with one or more embodiments.

FIG. 7 illustrates an example user interface that may be implemented in a pharmacy workflow management system in accordance with one or more embodiments.

FIG. 8 illustrates an example user interface that may be implemented in a pharmacy workflow management system in accordance with one or more embodiments.

FIG. 9 illustrates an example user interface that may be implemented in a pharmacy workflow management system in accordance with one or more embodiments.

FIG. 10 illustrates an example user interface that may be implemented in a pharmacy workflow management system in accordance with one or more embodiments.

FIG. 11 conceptually illustrates an electronic system with which one or more embodiments of the subject technology may be implemented.

### DETAILED DESCRIPTION

The detailed description set forth below is intended as a description of various configurations of the subject technology and is not intended to represent the only configurations in which the subject technology may be practiced. The appended drawings are incorporated herein and constitute a part of the detailed description. The detailed description includes specific details for the purpose of providing a thorough understanding of the subject technology. However, it will be clear and apparent to those skilled in the art that the subject technology is not limited to the specific details set forth herein and may be practiced using one or more embodiments. In one or more instances, well-known structures and components are shown in block diagram form in order to avoid obscuring the concepts of the subject technology.

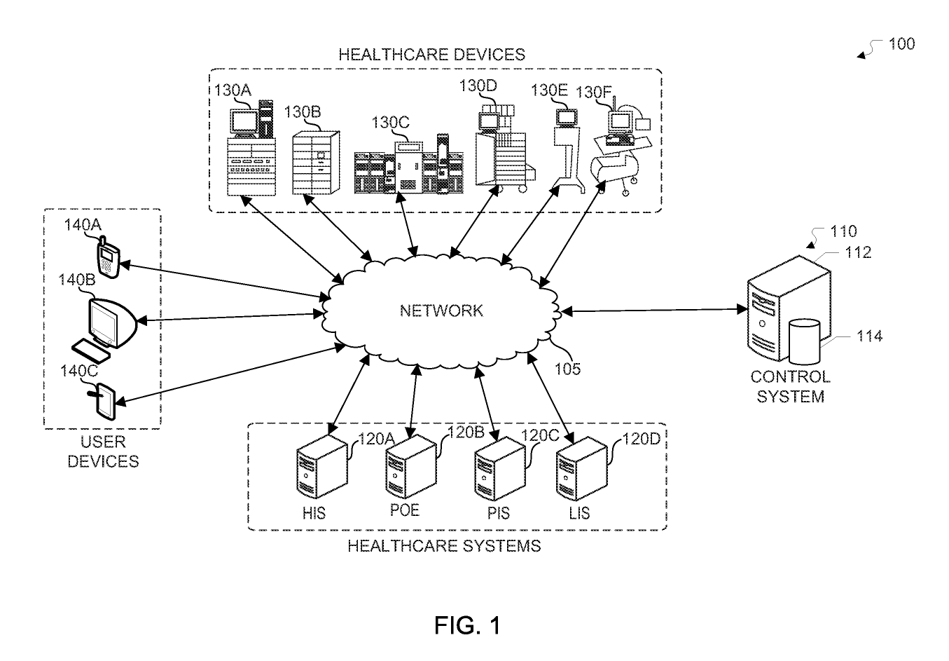

FIG. 1 illustrates an example network environment 100 in which a pharmacy workflow management system may be implemented in accordance with one or more embodiments. Not all of the depicted components may be required, however, and one or more embodiments may include additional components not shown in the figure. Variations in the arrangement and type of the components may be made without departing from the spirit or scope of the claims as set forth herein. Additional, different or fewer components may be provided.

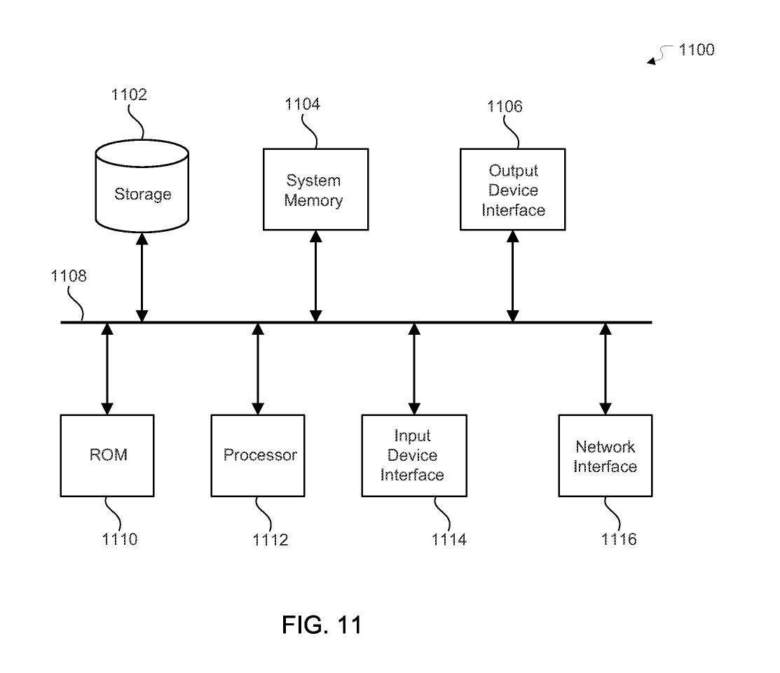

The network environment 100 includes network 105, a control system 110, one or more healthcare systems 120A-D, one or more healthcare devices 130A-F, and one or more user devices 140A-C. The control system 110, healthcare systems 120A-D, healthcare devices 130A-F, and/or user devices 140A-C may be communicatively coupled to one another, such as by the network 105. In one or more embodiments, one or more of the control system 110, healthcare systems 120A-D, healthcare devices 130A-F, or user devices 140A-C may be directly coupled to one another. In addition, there may be a number of other devices connected to the network 105, such as additional healthcare systems, e.g. other clinical and/or logistical systems, additional healthcare devices, external systems, computing devices, mobile devices, etc. The control system 110, one or more healthcare systems 120A-D, one or more healthcare devices 130A-F, and/or one or more user devices 140A-C may be, or may include all or part of, the electronic system that is discussed further below with respect to FIG. 11.

The network 105 may be a communication network, such as a public communication network (such as the Internet, cellular data network, dialup modems over a telephone network), a private communications network (such as private local area network (“LAN”), leased lines), etc. The network 105 may also include, but is not limited to, any one or more of the following network topologies, including a bus network, a star network, a ring network, a mesh network, a star-bus network, a tree or hierarchical network, and the like. The connections of the network 105 may be wired or wireless. For example, one or more of the control system 110, healthcare systems 120A-D, healthcare devices 130A-F, and/or user devices 140A-C may transmit wireless signals over the network 105, such as radio frequency (RF) signals, infrared (IR) signals, Bluetooth signals, or any other means capable of carrying information in a wireless manner between devices having appropriate transmitters and/or receivers.

The control system 110 may be a single computing device such as a computer server. Alternatively, the control system 110 may represent one or more computing devices (such as a cloud of computers and/or a distributed system) that are communicatively coupled, such as communicatively coupled over the network 105, and that collectively, or individually, perform one or more functions that can be performed server-side, such as receiving messages, transmitting messages, storing messaging, receiving control commands, providing user interfaces, transmitting notifications, etc. The one or more computing devices of the control system 110 may be geographically collocated and/or the one or more computing devices of the control system 110 may be disparately located. The control system 110 may be coupled with various databases, such as data store 114, storage services, or other computing devices. The control system 110, and the coupled databases, storage services, or other computing devices may be geographically collocated, or may be disparately located. In one or more embodiments, the control system 110 includes a processing device 112 and a data store 114. The processing device 112 executes computer instructions stored in the data store 114. In one or more embodiments, the data store 114 may store the computer instructions on non-transitory computer-readable medium.

The one or more healthcare systems 120A-D may be any systems that facilitate with providing healthcare, and/or provide healthcare. In FIG. 1, the healthcare system 120A is a hospital information system (HIS), the healthcare system 120B is a physician order entry (POE) system, the healthcare system 120C is a pharmacy information system (PIS), and the healthcare system 120D is a laboratory information system (LIS). The HIS may, for example, store information pertaining to the administration of the healthcare facility, such as a hospital. The HIS may provide, and/or may interface with a server that provides, billing and accounting functions. The POE system may be used, for example, by physicians to enter orders for patients, such as orders for medications to be administered to patients, that are then transmitted to the PIS.

The PIS may store, for example, information pertaining to a pharmacy of a healthcare facility, such as outstanding orders, filled orders, patient medical profiles/histories, etc. For example, the PIS may provide a library of drug allergies and adverse drug interactions against which each incoming order, or prescription, is checked as part of the order entering/drug dispensing process to identify possible allergies and adverse drug interactions and help in preventing administration of drugs to a patient where the patient might be injured by the prescribed course of therapy. Additionally, the PIS may check to determine if any therapies are being duplicated, such as where two or more drugs might be used to treat a diagnosed disease, whether they are synergistic or antagonistic, and whether the prescribed therapy should be modified accordingly. The LIS may store laboratory results, such as for tests performed to facilitate with providing healthcare to patients.

The healthcare devices 130A-F may include infusion devices, such as infusion pumps, drug delivery devices, dispensing devices, such as automated dispensing machines, smart beds, monitoring devices, respiratory devices, such as ventilators, waste devices, such as drug disposal devices, or generally any device that may facilitate with providing healthcare and/or may provide healthcare. The healthcare devices 130A-F may include a processor and memory. Alternatively, or in addition, the healthcare devices 130A-F may be communicatively coupled to a device that includes a processor and a memory, such as via a serial port.

For example, the healthcare devices 130A-F may include Pyxis Medstations™ to store and dispense medications at the nurses stations, providing distributed access to the medications needed to treat patients, Pyxis® Anesthesia Systems to store and manage the medications used by anesthesiologists in the operating room, Pyxis SpecialtyStations™ to store specific medications and supplies in individual treatment areas, and Pyxis OncologyStations™ in oncology departments to manage the specialized and hazardous medications used to treat cancer. The healthcare devices 130A-F may also include waste devices that accept and store wasted medications, e.g. excess medications, from healthcare professionals and track the amount of medications wasted by healthcare professionals. In one or more embodiments, one or more of the waste devices may be a Pyxis EcoStation™ system.

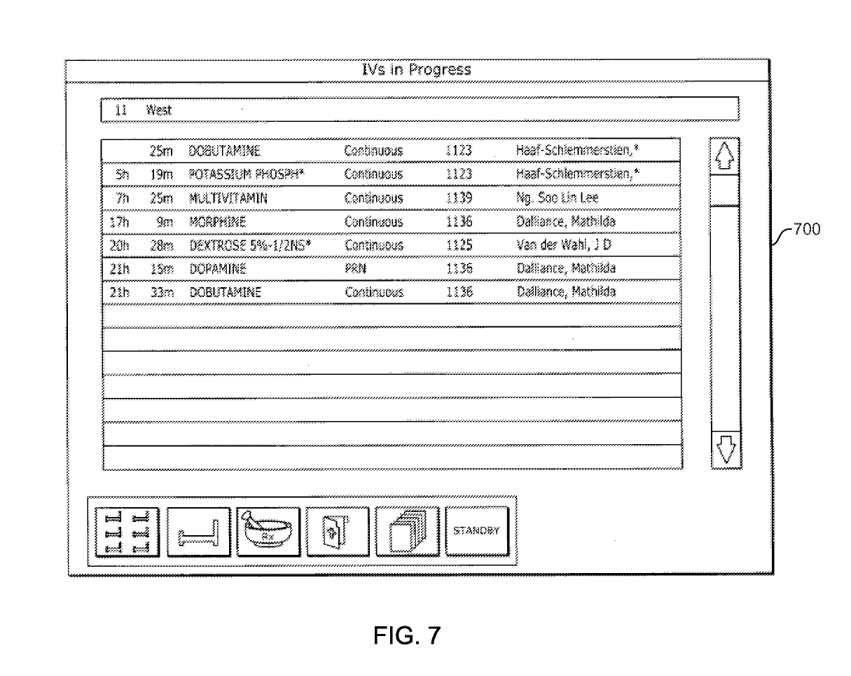

The user devices 140A-C may be electronic devices such as laptop or desktop computers, mobile phones, personal digital assistants (“PDAs”), portable media players, tablet computers, televisions or other displays, or other appropriate computing devices that can be used to display user interfaces that facilitate providing healthcare to patients, such as user interfaces that display information related to providing healthcare to patients and/or user interfaces that allow a healthcare professional, such as a doctor or nurse, access, create, and/or modify information related to providing healthcare to patients, such as modifying a schedule for preparing IVs in the PIS. Example user interfaces are discussed further below with respect to FIGS. 7-10. In the example of FIG. 1, the user device 140A is depicted as a mobile phone, the user device 140B is depicted as a desktop computer, and the user device 140C is depicted as a personal digital assistant (“PDA”), e.g. a tablet device. In one or more embodiments, the user devices 140A-C may include a processor and a memory.

In operation, the control system 110, the healthcare systems 120A-D, the healthcare devices 130A-F, and/or the user devices 140A-C may transmit electronic data streams to one another over the network 105. The electronic data streams may include, for example, messages, and one or more of the messages may be referred to as a medical transaction carrier (MTC). The messages may relate to healthcare that is being facilitated by any of the healthcare systems 120A-D, the healthcare devices 130A-F, and/or the user devices 140A-C. For example, a message may include an order for a medication that is transmitted from a POE system to a PIS. In one or more embodiments, at least a portion of the message, and/or a second message related to the message, may be transmitted by the PIS to a healthcare device 130A, such as to indicate that the ordered medication should be administered to the patient. Alternatively, or in addition, a message may relate to the progress of the delivery of medication, such as by one or more of the healthcare devices 130A-F. For example, the healthcare device 130A may transmit a message to the PIS that indicates the progress of delivering the medication by the healthcare device 130A to the patient. For example, the message may indicate that the healthcare device 130A has started delivering the medication, the healthcare device 130A has delivered an indicated amount of the medication, or the healthcare device 130A has completed the delivery of the medication.

The control system 110 and/or one of the healthcare systems 120A-D, such as the healthcare system 120C (PIS), may utilize the messages regarding the progress of delivering medications to manage a pharmacy workflow schedule. For example, an order for a medication that is being delivered to a patient may need to be refilled as soon as the delivery of the medication is complete. Thus, the healthcare system 120C may generate a pharmacy workflow schedule for preparing medications to ensure that any orders for medications that need to be refilled and/or any orders for medications that otherwise need to be prepared within certain time constraints, are available at the necessary times, e.g. when the delivery and/or administration of a medication completes. The healthcare system 120C may also generate the schedule to ensure that any orders for medications that are received from the healthcare system 120B (POE) and/or any of the other healthcare systems 120A,D, are prepared in a timely manner. In one or more embodiments, the healthcare system 120C may also generate the schedule to ensure that, for a continuous infusion, a refill or replacement bag is available slightly before the completion of a current bag, thereby ensuring that there is no interruption in the continuous infusion.

In one or more embodiments, the pharmacy workflow schedule may be regulated by a queue system that is fed with new orders, e.g. from the healthcare system 120B, change and/or refill orders, e.g. from the healthcare system 120B, and the real-time data received from the healthcare devices 130A-F. A pharmacy technician may utilize the queue system to determine what action to perform next with respect to compounding or drug preparation. In one or more embodiments, a pharmacy may be managed by one or more robots in combination with one or more pharmacy technicians. The pharmacy workflow may also be regulated by a series of different steps, which in some instances, may need to be verified by a pharmacist. Thus, the pharmacy technicians and the pharmacists may perform actions with a combination of automated and manual systems both to compound medications and to communicate the status of the compounding process. For example, a printer in the pharmacy may print out a label to indicate that an order needs fulfillment, and/or a robot may start compounding an order when necessary.

In one or more embodiments, the control system 110 and/or the healthcare system 120C, may modify the schedule as messages are received from the healthcare devices 130A-F regarding the progress of delivering and/or administering medications. For example, if a healthcare device 130A transmits a message indicating that delivery and/or administration of a medication (for which a refill is required) is running behind schedule, the control system 110 and/or the healthcare system 120C may modify the schedule such that the refill of the medication is delayed in accordance with the delay in the delivery and/or administration of the medication. Similarly, if a healthcare device 130A transmits a message indicating that the infusion rate of a medication (for which a refill is required) has been increased, the control system 110 and/or the healthcare system 120C may modify the schedule such that the refill of the medication is available at an earlier time, in accordance with the increased infusion rate. In this manner, the pharmacy will not expend resources on preparing an order before it is required, and the pharmacy can therefore reallocate any such resources to the preparation of other medications.

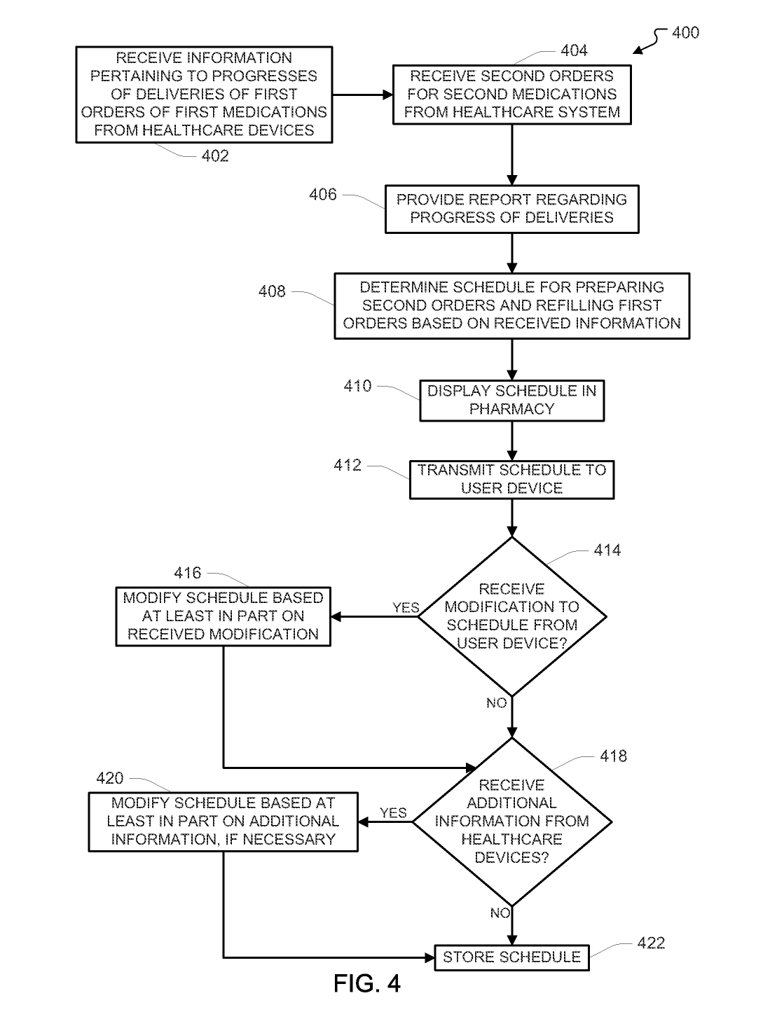

Alternatively, or in addition, if a healthcare device 130A is an automated drug dispensing device, the healthcare device 130A may transmit a message to the healthcare system 120C when a medication dispensed by the healthcare device 130A is running low on stock. Thus, the healthcare system 120C can add the preparation of the medication that is low on stock to the pharmacy workflow schedule. An example process for a pharmacy workflow management system is discussed further below with respect to FIG. 4.

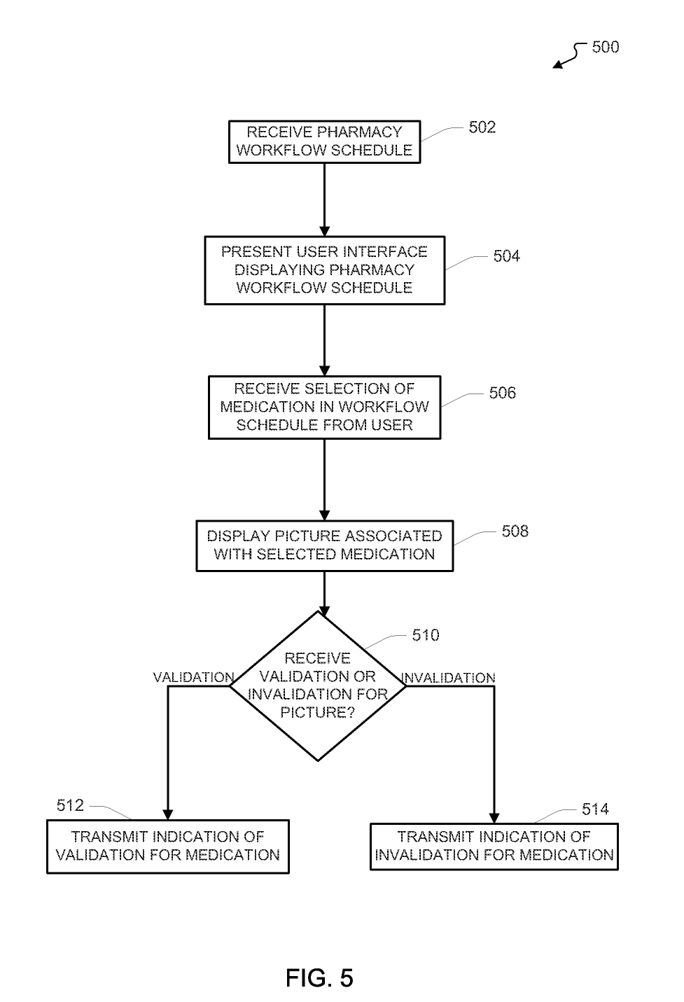

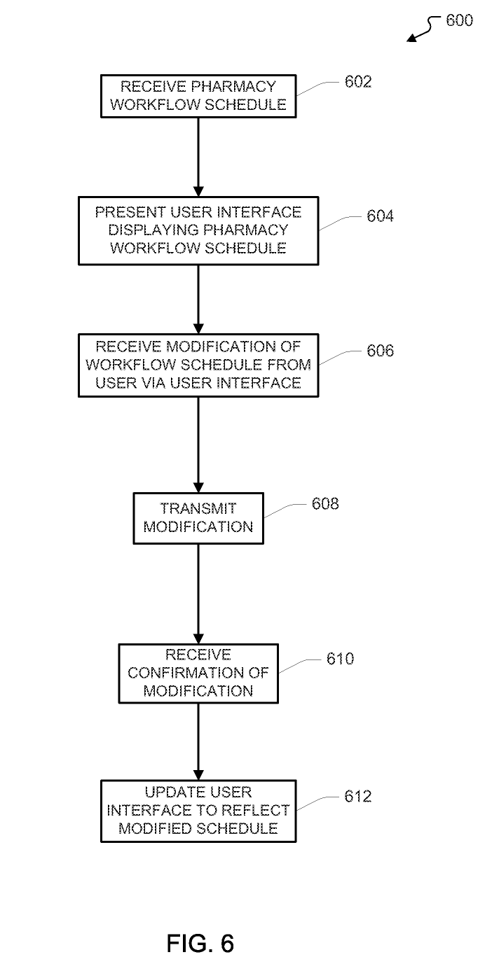

In one or more embodiments, the control system 110 and/or the healthcare system 120C may provide information related to the pharmacy workflow to one or more of the user devices 140A-C, such as the user device 140A. For example, the control system 110 and/or the healthcare system 120C may provide the statuses of all medications being delivered to patients, e.g. infusions, and/or the control system 110 and/or the healthcare system 120C may provide a schedule of the pharmacy workflow, e.g. the schedule/status of medications being delivered, being prepared, and/or scheduled to be prepared. In the instance of a schedule of the pharmacy workflow, the user device 140A may display the schedule to a user, such as a healthcare professional. In one or more embodiments, the healthcare professional may interact with the user interface to select one of the items listed on the schedule, such as a medication that has been compounded and is being delivered to a patient. In response to the selection, the user device 140A may display a picture of the medication that is being delivered to the patient on the user interface. The healthcare professional may review the picture and either provide an indication that the medication being delivered to the patient is the correct medication, e.g. validate the medication, or provide an indication that the medication being delivered to the patient is incorrect, e.g. invalidate the medication. In one or more embodiments, a healthcare professional may interact with a user device 140A that is displaying a pharmacy workflow schedule to modify the schedule. For example, the healthcare professional may modify the schedule to cancel the preparation of a medication, expedite the preparation of a medication, delay the preparation of a medication, or generally to perform any necessary rescheduling of the pharmacy workflow. Example processes for user devices 140A-C in a pharmacy workflow management system are discussed further below with respect to FIGS. 5 and 6.

The control system 110 may provide user identity and notification systems. For example, the control system 110 may authenticate users and may control the access of a user, or a group of users. For example, a physician may be allowed to input orders into a POE system, while a nurse may only be allowed to view the orders in the POE system. Thus, the control system 110 may provide different views of information, e.g. information received from one or more of the healthcare systems 120A-D and/or the healthcare devices 130A-F, to different users based on the users' access privileges. The control system 110 may also provide user interfaces to users, such as via the user devices 140A-C, and may manage the users' interactions with the user interfaces. In one or more embodiments, the control system 110 may provide information for a patient to a user device 140A to be displayed on one of the user interfaces when the control system 110 determines that the user device 140A is within a proximity of the patient and/or within a proximity of one of the healthcare devices 130A-F that is providing, and/or facilitating with providing, healthcare to the patient.

Alternatively, or in addition, the user device 140A may determine that it is located within a proximity of a patient and/or within a proximity of one of the healthcare devices 130A-F. The user device 140A may then transmit an indication to the control system 110 indicating that the user device 140A is located within the proximity of the patient and/or the one of the healthcare devices 130A-F. In response thereto, the control system 110 may transmit information to the user device 140A that pertains to the patient and/or the one of the healthcare devices 130A-F.

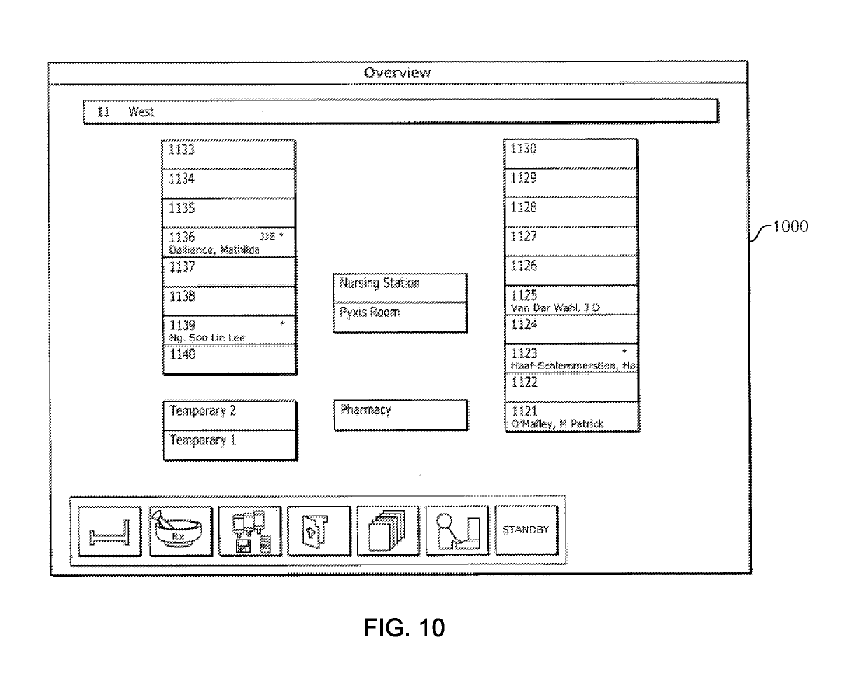

The control system 110 may also transmit notifications to one or more of the users, such as via the user devices 140A-C. For example, the control system 110 may transmit a notification to a user device 140A being accessed by a user, e.g. a user device 140A that the user has authenticated on, when the control system 110 determines that the user is within proximity of a patient who may need care, e.g. a patient that is receiving healthcare from one of the healthcare devices 130A-F that may be experiencing an error. In one or more embodiments, one or more of the notifications may be transmitted to the user devices 140A-C via a user interface. For example, the notification may cause a graphical indicator to be presented on a user interface being displayed on a user device 140A. Example user interfaces for presenting notifications are discussed further below with respect to FIGS. 7 and 10.

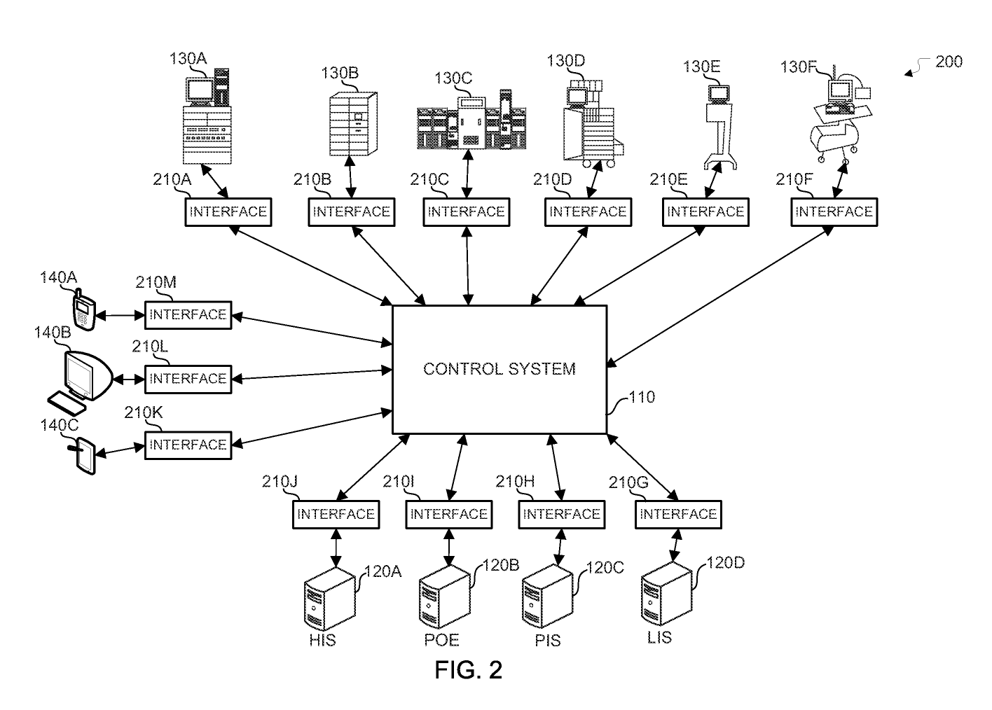

FIG. 2 illustrates an example messaging architecture 200 in which a pharmacy workflow management system may be implemented in accordance with one or more embodiments. Not all of the depicted components may be required, however, and one or more embodiments may include additional components not shown in the figure. Variations in the arrangement and type of the components may be made without departing from the spirit or scope of the claims as set forth herein. Additional, different or fewer components may be provided.

The messaging architecture 200 includes the control system 110, one or more healthcare systems 120A-D, one or more healthcare devices 130A-F, and one or more user devices 140A-C. The control system 110, healthcare systems 120A-D, healthcare devices 130A-F, and user devices 140A-C may be communicably coupled to one another, such as by the network 105 shown in FIG. 1. The one or more healthcare systems 120A-D, one or more healthcare devices 130A-F, and one or more user devices 140A-C may include, and/or may be coupled to, interfaces 210A-M. The interfaces 210A-M may be adapters that are utilized by the one or more healthcare systems 120A-D, one or more healthcare devices 130A-F, and one or more user devices 140A-C to transmit messages to one another via the control system 110. In one or more embodiments, the interfaces 210A-M may be, and/or may include, the adapters described in U.S. patent application Ser. No. 13/421,776, entitled “Scalable Communication System,” filed on Mar. 15, 2012, which is hereby incorporated by reference in its entirety for all purposes.

In one or more embodiments, messages transmitted by the healthcare systems 120A-D, the healthcare devices 130A-F, and/or the user devices 140A-C may be routed through the control system 110, e.g. via the interfaces 210A-M. For example, if a healthcare device 130A is sending a message to the healthcare system 120C, the healthcare device 130A may utilize the interface 210A to transmit the message to the control system 110, and the control system 110 may forward the message to the interface 210H, which provides the message to the healthcare system 120C. In one or more embodiments, the control system 110 may store the messages, such as in the data store 114, for further processing, such as to identify whether to transmit any information indicated in the messages to one or more of the user devices 140A-C, such as via a notification and/or via a user interface.

In one or more embodiments, the control system 110 may include an interface system that receives the messages from one or more of the healthcare systems 120A-D, the healthcare devices 130A-F, and/or the user devices 140A-C, via the interfaces 210A-M. The interface system may provide the interfaces 210A-M to the one or more of the healthcare systems 120A-D, the healthcare devices 130A-F, and the user devices 140A-C, and the one or more of the healthcare systems 120A-D, the healthcare devices 130A-F, and the user devices 140A-C may transmit messages to the interface system by utilizing the interfaces 210A-M.

In one or more embodiments, the interface system receives the messages in a first external format, e.g. a format native to the transmitting device and/or system, converts the messages into an internal messaging format, e.g. for processing and storing the messages, converts the messages into a second external format, e.g. a format native to the receiving device and/or system, and then transmits the messages in the second external format to the receiving device. In one or more embodiments, the first external format may be the same as the second external format. The interface system may be implemented as described, for example, in U.S. patent application Ser. No. 13/421,776, entitled “Scalable Communication System,” filed on Mar. 15, 2012, which has been incorporated by reference in its entirety for all purposes.

Alternatively, or in addition, one or more of the healthcare systems 120A-D, the healthcare devices 130A-F, and/or the user devices 140A-C may communicate with the control system 110 without utilizing the interfaces 210A-M. Alternatively, or in addition, one or more of the healthcare systems 120A-D, the healthcare devices 130A-F, and/or the user devices 140A-C may transmit messages directly to one another, e.g. without routing the messages through the control system 110.

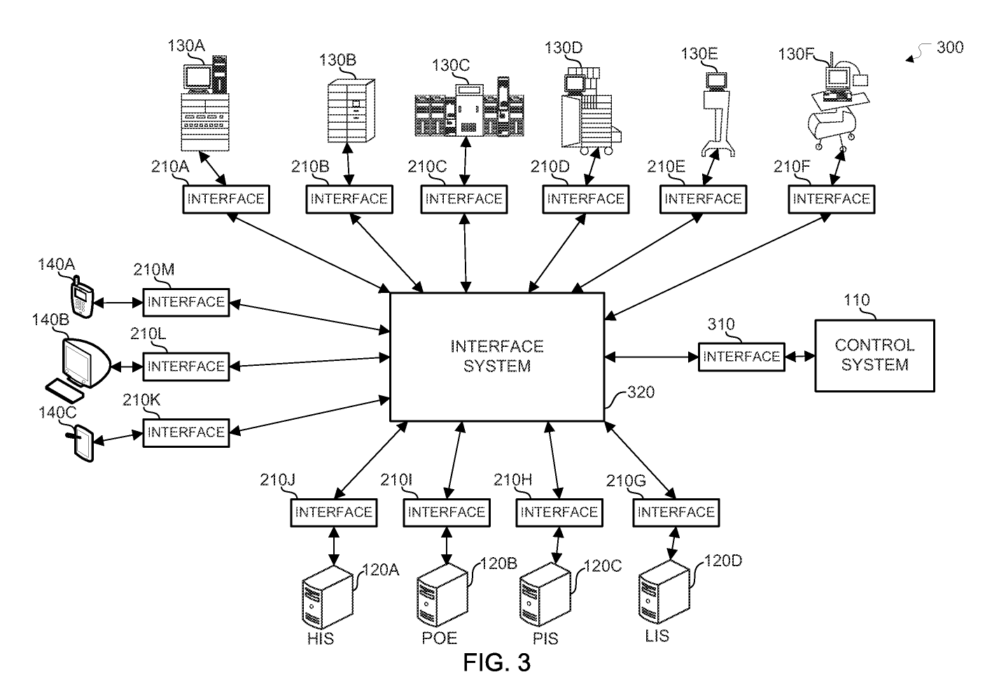

FIG. 3 illustrates an alternative example messaging architecture 300 in which a pharmacy workflow management system may be implemented in accordance with one or more embodiments. Not all of the depicted components may be required, however, and one or more embodiments may include additional components not shown in the figure. Variations in the arrangement and type of the components may be made without departing from the spirit or scope of the claims as set forth herein. Additional, different or fewer components may be provided.

The messaging architecture 300 includes an interface system 320, the control system 110, one or more healthcare systems 120A-D, one or more healthcare devices 130A-F, and one or more user devices 140A-C. The control system 110, interface system 320, healthcare systems 120A-D, healthcare devices 130A-F, and user devices 140A-C may be communicably coupled to one another, such as by the network 105 shown in FIG. 1. The one or more healthcare systems 120A-D, one or more healthcare devices 130A-F, and one or more user devices 140A-C may include, and/or may be coupled to, interfaces 210A-M. The control system 110 may include, and/or may be communicatively coupled to, the interface 310. The interfaces 210A-M, 310 may be adapters that are utilized by the one or more healthcare systems 120A-D, one or more healthcare devices 130A-F, one or more user devices 140A-C, and control system 110 to transmit messages to one another via the interface system 320. In one or more embodiments, the interfaces 210A-M, 310 may be, and/or may include, the adapters described in U.S. patent application Ser. No. 13/421,776, entitled “Scalable Communication System,” filed on Mar. 15, 2012, which was previously incorporated by reference in its entirety for all purposes.

In the messaging architecture 300, the interface system 320 may be separate from the control system 110, e.g. such that messages to/from the control system 110 are routed through the interface system 320. For example, the control system 110 and the interface system 320 may be separate devices, such as separate servers, or the control system 110 and the interface system 320 may be and/or may include distinct hardware on the same device. Alternatively, the control system 110 may receive messages directly from the interface system 320, e.g. without the use of the interface 310. Thus, in the messaging architecture 300, messages are routed through the interface system 320, rather than through the control system 110, as previously discussed with respect to FIG. 2.

Alternatively, or in addition, one or more of the healthcare systems 120A-D, the healthcare devices 130A-F, the user devices 140A-C, and/or the control system 110 may communicate with the interface system 320 without utilizing the interfaces 210A-M, 310. Alternatively, or in addition, one or more of the healthcare systems 120A-D, the healthcare devices 130A-F, the user devices 140A-C, and/or the control system 110 may transmit messages directly to one another, e.g. without routing the messages through the interface system 320.

FIG. 4 illustrates a flow diagram of an example process 400 for a pharmacy workflow management system in accordance with one or more embodiments. For explanatory purposes, the example process 400 is described herein with reference to the healthcare system 120C (PIS) of the example network environment 100 of FIG. 1; however, the example process 400 is not limited to the healthcare system 120C of the example network environment 100 of FIG. 1. For example, in one or more embodiments the example process 400 may be performed by the control system of FIG. 1, any of the healthcare systems 120A, B, D of FIG. 1, and/or the interface system 320 of FIG. 3. Further for explanatory purposes, the blocks of the example process 400 are described herein as occurring in serial fashion, or linearly. However, multiple blocks of the example process 400 may occur in parallel. In addition, the blocks of the example process 400 need not be performed in the order shown and/or one or more of the blocks of the example process 400 need not be performed.

In block 402, the healthcare system 120C receives information pertaining to the progresses of the deliveries of first orders for first medications from one or more of the healthcare devices 130A-F. For example, the healthcare devices 130A-F may transmit messages over the network 105 to the healthcare system 120C. The messages may indicate the progress of the delivery of a medication to a patient, such as a message that indicates that the medication has been received, e.g. an IV bag has been hung, a message that indicates that the delivery of the medication has been initiated, a message that indicates that a certain amount of the medication has been delivered, a message that indicates that the delivery of the medication is complete, and/or a message that generally indicates any information regarding the progress of the delivery of a medication. In one or more embodiments, an order for a medication that is being delivered may need to be refilled when the delivery of the medication is complete. Thus, the refill of the order for the medication should be prepared and accessible to a healthcare professional at the time that the delivery of the medication completes. In block 404, the healthcare system 120C (PIS) receives second orders for second medications from the healthcare system 120B (POE). For example, the healthcare system 120B may receive orders for medications, such as from a physician, and the healthcare system 120B may transmit the orders over the network 105 to the healthcare system 120C. In one or more embodiments, any of the first medications and any of the second medications may be the same medication or may be different medications.

In block 406, the healthcare system 120C may provide a report regarding the progress of the deliveries, such as to one of the user devices 140A-C over the network 105, and/or on a screen or display, such as in a pharmacy. An example user interface displaying a report regarding the statuses of in-progress deliveries of medications is discussed further below with respect to FIG. 7.

In block 408, the healthcare system 120C determines a schedule for preparing the second orders received from the healthcare system 120B, and for preparing any refills with respect to the in-progress deliveries of medications. In one or more embodiments, the pharmacy workflow schedule may also be referred to as a queue, and/or the pharmacy workflow schedule may include one or more queues, such as an IV compounding queue. The healthcare system 120C may prepare the schedule such that any refills that need to be prepared with respect to in-progress deliveries will be prepared and accessible to a healthcare professional at the time that the deliveries complete. The healthcare system 120C may also prepare the schedule such that the second orders are prepared in a timely fashion, and/or such that any timing requirements with respect to preparing the second orders are met.

In block 410, the healthcare system 120C displays the schedule in a pharmacy. For example, the healthcare system 120C may display the schedule on a screen or monitor in the pharmacy that is communicatively coupled to the healthcare system 120C, and/or healthcare system 120C may transmit the schedule to a device in the pharmacy that includes a screen and/or monitor. In one or more embodiments, the healthcare system 120C may combine the schedule with the report of the in-progress deliveries to display a dashboard showing all in-progress deliveries of medications, the progress of medications being prepared (e.g. in preparation, provided to healthcare professional, etc.), and the schedule of medications to be prepared. In one or more embodiments, devices displaying the dashboard may be mounted in the pharmacy work areas, including the IV compounding area, nursing areas, or generally any areas of a healthcare facility. The dashboard may be used by pharmacy technicians or clinical staff responsible for reviewing or compounding IV orders. In one or more embodiments, the dashboard may serve as an information source and guide for a clinical pharmacist that approves the refill of IV orders, in addition to a guide for technicians performing the IV compounding. For example, pharmacy staff may utilize the dashboard to compare the status of in-progress IVs to IV orders being prepared in order to prevent IV compounding for orders that have been discontinued. Similarly, pharmacy staff may utilize the dashboard to escalate the priority of IV compounding orders that are urgently needed in patient care. In one or more embodiments, the healthcare system 120C may update the dashboard in “real-time”, e.g. as messages are received from the healthcare devices 130A-F and/or the healthcare system 120B.

In block 412, the healthcare system 120C transmits the pharmacy workflow schedule to one or more of the user devices 140A-C, such as the user device 140A. For example, the healthcare system 120C may transmit the pharmacy workflow schedule to the user device 140A over the network 105. The user device 140A may display the schedule to a healthcare professional, such as a pharmacist, clinician, physician, etc. In one or more embodiments, the healthcare professional may interact with the user device 140A to modify the schedule. For example, a pharmacist may review the priority of an order on the existing schedule and, based on new clinical information, change the priority of the order, e.g. by interacting with the user interface being displayed by the user device 140A. Similarly, a pharmacist may eliminate an order from the schedule that is no longer needed, thereby preventing IV waste. In response to a healthcare professional interacting with the user interface to modify the schedule, the user device 140A may transmit an indication of the modification to the healthcare system 120C, such as over the network 105.

Alternatively, or in addition, the healthcare system 120C may transmit notifications to one or more user devices 140A-C of healthcare professionals that are associated with orders that are listed in the pharmacy workflow schedule. For example, the healthcare system 120C may transmit notifications to user devices 140A-C of nurses that are associated with orders listed in the pharmacy workflow schedule in order to provide the nurses with some insight into how the preparation of the medications is progressing.

In block 414, the healthcare system 120C determines whether it received any indications of modifications of the workflow management schedule from any of the user devices 140A-C, such as the user device 140A. If, in block 414, the healthcare system 120C determines that it received an indication of a modification from the user device 140A, then the healthcare system 120C moves to block 416. In block 416, the healthcare system 120C modifies the schedule based at least in part on the received modification from the user device 140A. The healthcare system 120C may transmit a confirmation of the modification to the user device 140A, in addition to any other devices that are displaying the schedule. In one or more embodiments, the healthcare system 120C may validate, or verify, the modification before modifying the schedule, such as by applying the modification to one or more business rules.

If, in block 414, the healthcare system 120C determines that it has not received any indications of modifications from any of the user devices 140A-C, then the healthcare system 120C moves to block 418. In block 418, the healthcare system 120C determines whether it has received any additional messages regarding the progress of deliveries of medications from any of the healthcare devices 130A-F. If, in block 418, the healthcare system 120C determines that it has received additional information from at least one of the healthcare devices 130A-F, such as the healthcare device 130A, the healthcare system 120C moves to block 420. In block 420, the healthcare system 120C processes the received message and determines whether a modification to the schedule is necessary in view of the received message. For example, if the received message indicates that an order has been cancelled, the healthcare system 120C may modify the schedule to remove the preparation of the cancelled order. In one or more embodiments, the healthcare system 120C may transmit any modifications to the schedule to any user devices 140A-C, and/or other monitors/displays, that have previously received the schedule.

If, in block 418, the healthcare system 120C determines that it has not received additional information from any of the healthcare devices 130A-F, then the healthcare system 120C moves to block 422. In block 422, the healthcare system 120C stores the schedule, such as in a data store. For example, the healthcare system 120C may store the schedule in a local data store and/or a remote data store, such as the data store 114.

FIG. 5 illustrates a flow diagram of an example process 500 for a user device 140A in a pharmacy workflow management system in accordance with one or more embodiments. For explanatory purposes, the example process 500 is described herein with reference to the user device 140A and the healthcare system 120C of the example network environment 100 of FIG. 1; however, the example process 500 is not limited to the user device 140A and/or the healthcare system 120C of the example network environment 100 of FIG. 1. Further for explanatory purposes, the blocks of the example process 500 are described herein as occurring in serial fashion, or linearly. However, multiple blocks of the example process 500 may occur in parallel. In addition, the blocks of the example process 500 need not be performed in the order shown and/or one or more of the blocks of the example process 500 need not be performed.

In block 502, the user device 140A may receive a pharmacy workflow schedule. For example, the user device 140A may receive a pharmacy workflow schedule from the healthcare system 120C over the network 105. The pharmacy workflow schedule may list one or more of medications scheduled for preparation, medications being prepared, medications provided to healthcare professionals, such as via an automated dispensing machine, medications being delivered to patients, and/or medications for which delivery is completed.

In block 504, the user device 140A may present a user interface displaying the pharmacy workflow schedule, such as on a screen to a healthcare professional. The pharmacy workflow schedule may include graphical indicators, such as delineated rows, that correspond to one or more of the medications listed in the pharmacy workflow schedule. In one or more embodiments, a user may be able to select a medication in the schedule by selecting the corresponding graphical indicator. In block 506, the user device 140A receives a selection of one of the medications listed in the pharmacy workflow schedule from a user, such as a healthcare professional. For example, a healthcare professional may select the graphical indicator associated with a medication on the user interface.

In block 508, the user device 140A may display a picture that is associated with the selected medication. For example, in response to receiving the selection of the medication the user device 140A may request the picture from the healthcare system 120C. The healthcare system 120C may then transmit the picture to the user device 140A, and the user device 140A display the picture. In one or more embodiments, the healthcare system 120C may store and provide pictures of the medications as they progress through each step of the pharmacy workflow. For example, the healthcare system 120C may store a picture of a medication as it is being compounded, a picture of a medication as it is being provided to a healthcare professional, a picture of a medication as it is being delivered to a patient, or generally a picture of a medication at any point in the workflow. In one or more embodiments, the pictures may be validated in order to confirm that the medication is being prepared and/or delivered properly. For example, the pictures may be validated by a computing device, such as using a picture recognition algorithm, and/or validated by a user, such as by a healthcare professional who is interacting with the user device 140A. For example, a picture recognition algorithm may be able to read a label on a medication in a picture and compare the label to the expected result for the particular step.

In one or more embodiments, a user may configure the healthcare system 120C to identify which medications can be validated using the automated algorithm and which medications need to be validated by a healthcare professional.

In block 510, the user device 140A determines whether the user provided a validation of the displayed picture, e.g. of the preparation and/or delivery of the medication. For example, the user interface that displays the picture may also include one or more selectors, such as buttons, for indicating, e.g. that the preparation and/or delivery of the medication, as indicated by the picture, has been validated or invalidated. If, in block 510, the user device 140A receives an indication from the user that the picture has been validated, the user device 140A moves to block 512. In block 512, the user device 140A transmits an indication of the validation to the healthcare system 120C, such as over the network 105. The healthcare system 120C may store the validation, such as in a data store.

If, in block 510, the user device 140A receives an indication from the user that the picture was invalidated, the user device 140A moves to block 514. In block 514, the user device 140A transmits an indication of the invalidation to the healthcare system 120C, such as over the network 105. The healthcare system 120C may store the invalidation, such as in a data store. Alternatively, or in addition, the healthcare system 120C may transmit one or more notifications regarding the invalidation. For example, the healthcare system 120C may identify a display and/or one or more user devices 140A-C that are located within a proximity of the location where the picture was taken, e.g. the location where the invalid process is occurring. The healthcare system 120C may then transmit a notification, such as an alert, to the display and/or the one or more user devices 140A-C that indicates that corrective action is required. In one or more embodiments, upon receiving an indication of invalidation from the user, the user device 140A may provide the user with a user interface for controlling and/or modifying the aspect of the process that was invalidated. The user may interact with the user device 140A, e.g. via the user interface, to perform a corrective action with respect to the invalidated process.

FIG. 6 illustrates a flow diagram of an example process 600 for a user device 140A in a pharmacy workflow management system in accordance with one or more embodiments. For explanatory purposes, the example process 600 is described herein with reference to the user device 140A of the example network environment 100 of FIG. 1; however, the example process 600 is not limited to the user device 140A of the example network environment 100 of FIG. 1. Further for explanatory purposes, the blocks of the example process 600 are described herein as occurring in serial fashion, or linearly. However, multiple blocks of the example process 600 may occur in parallel. In addition, the blocks of the example process 600 need not be performed in the order shown and/or one or more of the blocks of the example process 600 need not be performed.

In block 602, the user device 140A receives a pharmacy workflow schedule. For example, the user device 140A may receive a pharmacy workflow schedule from the healthcare system 120C. The pharmacy workflow schedule may list one or more of medications scheduled for preparation, medications being prepared, medications provided to healthcare professionals, such as via an automated dispensing machine, medications being delivered to patients, and/or medications for which delivery is completed.

In block 604, the user device 140A presents a user interface displaying the pharmacy workflow schedule, such as to a healthcare professional. The pharmacy workflow schedule may include graphical indicators for one or more of the medications listed in the pharmacy workflow schedule. In one or more embodiments, a user may be able to select a medication in the schedule by selecting the corresponding graphical indicator. In block 606, the user device 140A may receive a modification of the schedule, such as from a user interacting with the user device 140A via the user interface. For example, a user may select a medication in the schedule by selecting the corresponding graphical indicator. Upon selecting a medication in the schedule, the user may change the scheduled preparation of the selected medication, such as by cancelling the preparation, increasing the priority of the preparation, decreasing the priority of the preparation, etc.

In block 608, the user device 140A may transmit the modification to the healthcare system 120C. For example, the user device 140A may transmit the modification over the network 105 to the healthcare system 120C. In one or more embodiments, the healthcare system 120C may receive the modification and may verify whether the modification is permissible and/or appropriate, such as by applying one or more business rules against the modification. If the healthcare system 120C determines that the modification is permissible, the healthcare system 120C may modify the schedule based at least in part on the received modification and may transmit a confirmation of the modification to the user device 140A, in addition to any other devices that have received the schedule.

In block 610, the user device 140A receives a confirmation of the modification from the healthcare system 120C. For example the user device 140A may receive the confirmation from the healthcare system 120C over the network 105. In block 612, the user device 140A updates the schedule displayed on the user interface to reflect the confirmed modification to the schedule. The user device 140A may then receive additional modifications from a user via the user interface.

FIG. 7 illustrates an example user interface 700 that may be implemented in a pharmacy workflow management system in accordance with one or more embodiments. Not all of the depicted components may be required, however, and one or more embodiments may include additional components not shown in the figure. Variations in the arrangement and type of the components may be made without departing from the spirit or scope of the claims as set forth herein. Additional, different or fewer components may be provided.

The user interface 700 may display information relating to in-progress actions being performed by one or more of the healthcare devices 130A-F, such as medical orders being administered. For example, the user interface 700 displays a report of IVs that are being administered by, and/or with the facilitation of, one or more of the healthcare devices 130A-F. In one or more embodiments, the administrations of medical orders that will terminate within a preselected time period may be distinguished on the user interface 700 from other administrations by color highlighting or other means. The user interface 700 may further display the time remaining, medication, and patient name, as well as buttons for program control. In one or more embodiments, the user interface 700 may display pending infusions or infusions scheduled to begin within a preselected time period.

In operation, the user interface 700 may be provided by the control system 110, and/or one or more of the healthcare systems 120A-D, for display on a screen, such as a screen of one or more of the user devices 140A-C, and/or a screen or monitor associated with one or more of the healthcare systems 120A-D. For example, the control system 110 may receive messages from one or more of the healthcare devices 130A-F related to actions being performed by the one or more healthcare devices 130A-F. The control system 110 may parse the received messages to obtain the information displayed on the user interface 700, and/or the received messages may be displayed on the user interface 700.

The information displayed on the user interface 700 may be updated in real-time as the control system 110 and/or one or more of the healthcare systems 120A-D receives messages from the healthcare devices 130A-F, such as while orders are being administered to patients. In one or more embodiments, the user interface 700 may be used to modify the preparation of medications, such as by scheduling and/or rescheduling, the preparation of medications.

In one or more embodiments, the user interface 700 may also display notifications, and/or alerts, such as when a healthcare professional is associated with a user device 140A that is displaying the user interface 700. For example, in the user interface 700, the notifications may be indicated by an asterisk (“*”). In one or more embodiments, the healthcare professional may select information that is displayed with an asterisk, such as by touching or clicking on the information, to receive additional information regarding the notification. In one or more embodiments, one or more of the alerts and/or notifications may only be displayed when the healthcare professional is proximally located to the one or more healthcare devices 130A-F to which the alerts and/or notifications pertain.

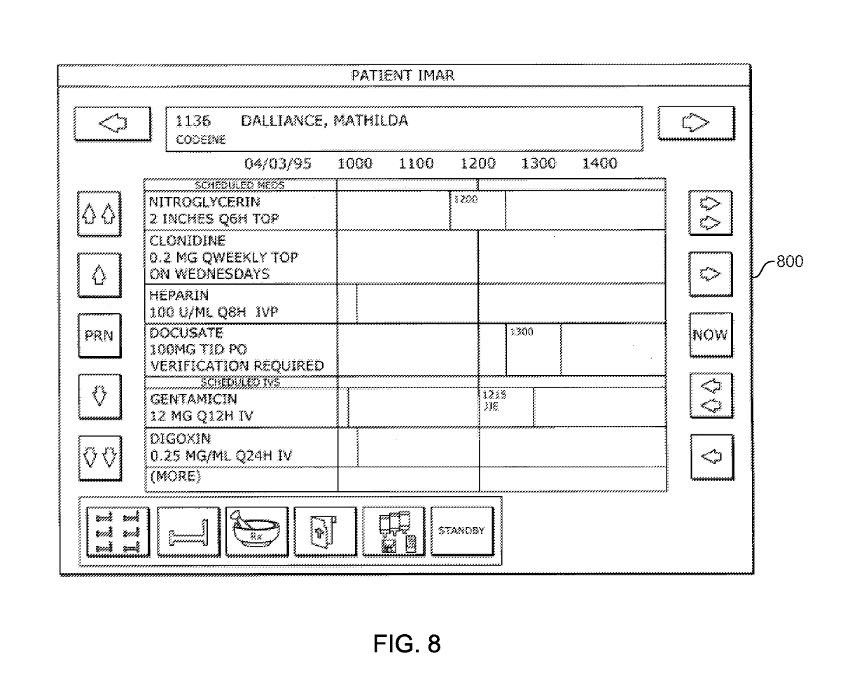

FIG. 8 illustrates an example user interface 800 that may be implemented in a pharmacy workflow management system in accordance with one or more embodiments. Not all of the depicted components may be required, however, and one or more embodiments may include additional components not shown in the figure. Variations in the arrangement and type of the components may be made without departing from the spirit or scope of the claims as set forth herein. Additional, different or fewer components may be provided.

The user interface 800 may display information relating to a patient, such as information received from one or more of the healthcare systems 120A-C and/or one or more of the healthcare devices 130A-F. The displayed information may include information related to medications and/or infusions, such as IVs, that are scheduled for the patient, and the information may be received from the healthcare system 120C. The information may further include information related to medications and/or infusions that are being administered to the patient and this information may be received from one or more of the healthcare devices 130A-F. For example, the user interface 800 displays information related to scheduled medications and IVs for an identified patient. In one or more embodiments, the user interface 800 may be color coded to indicate the status and schedule of each medication administration. For example, a medication delivery window extending from thirty minutes prior and thirty minutes after the scheduled administration time may be indicated by a yellow band on the user interface 800.

In operation, the user interface 800 may be provided by the control system 110, and/or one or more of the healthcare systems 120A-D, for display on a screen, such as a screen of one or more of the user devices 140A-C, and/or a screen or monitor associated with one or more of the healthcare systems 120A-D. For example, the control system 110 may receive messages from one or more of the healthcare systems 120A-D, and/or one or more of the healthcare devices 130A-F, that relate to a patient. The control system 110 may parse the received messages to obtain the information displayed on the user interface 800, and/or the received messages may be displayed on the user interface 800.

In one or more embodiments, the user interface 800 may automatically be provided to the user device 140A of a healthcare professional when the healthcare professional is located within a proximity of the patient and/or within a proximity of one or more healthcare devices 130A-F that are providing, and/or facilitating with providing, healthcare to the patient. Thus, the user device 140A of a healthcare professional may automatically display, and/or prepare for display, information related to an identified patient when the healthcare professional is within proximity of the patient, such as at the bedside of the patient. Accordingly, the information displayed on the user interface 800 may change as the healthcare professional moves throughout the healthcare facility.

In one or more embodiments, the user device 140A may receive information for displaying the user interface 800 when the healthcare professional is proximally located to the patient, but the user device 140A may not display the user interface 800 until prompted by the healthcare professional. For example, the user device 140A may receive the information for displaying the user interface 800 and may then display a notification to the healthcare professional indicating that the information is available, and requesting whether the healthcare professional would like the user interface 800 to be displayed on the user device 140A and/or on a display or monitor proximally located to the user device 140A.

The information displayed on the user interface 800 may be updated in real-time as the control system 110 and/or one or more of the healthcare systems 120A-D receives messages from the healthcare devices 130A-F, such as while orders are being administered to the patient. In one or more embodiments, the user interface 800 may be used to schedule and/or reschedule, the preparation of medications, such as by the healthcare system 120C.

The information displayed on the user interface 800 may be updated in real-time as the control system 110 and/or one or more of the healthcare systems 120A-D receives messages from the healthcare devices 130A-F and/or the healthcare systems 120A-D, such as while orders are being administered to patients, while orders are being prepared for administration to patients, and/or when orders are received from a physician order entry system. In one or more embodiments, the user interface 800 may be used to verify the administration of orders, such as a healthcare professional verifying that a medication scheduled for administration and/or a medication being administered coincides with the ordered medication. In one or more embodiments, a healthcare professional may be able to select, such as touch or click on, an order and the user interface 800 may display a picture of the medication being administered, or about to be administered.

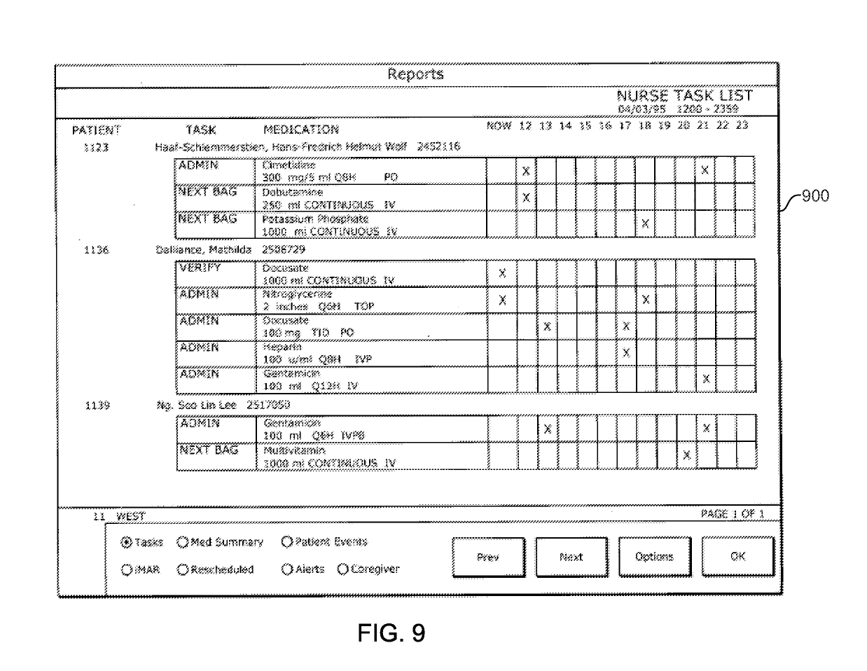

FIG. 9 illustrates an example user interface 900 that may be implemented in a pharmacy workflow management system in accordance with one or more embodiments. Not all of the depicted components may be required, however, and one or more embodiments may include additional components not shown in the figure. Variations in the arrangement and type of the components may be made without departing from the spirit or scope of the claims as set forth herein. Additional, different or fewer components may be provided.

The user interface 900 may display information to an identified healthcare professional that relates to in-progress actions being performed by, and/or with the facilitation of, one or more of the healthcare devices 130A-F, and for which the healthcare professional is facilitating and/or monitoring. The user interface 900 may also display information to the identified healthcare professional that relates to future actions to be performed by, and/or with the facilitation of, one or more of the healthcare devices 130A-F, and for which the healthcare professional is facilitating and/or monitoring. Alternatively, or in addition, the user interface 900 may display information related to in-progress or future actions that are being performed proximally to the user device 140A, and a healthcare professional accessing the user device 140A. For example, the user interface 900 displays a report of IVs that are being administered by one or more of the healthcare devices 130A-F. In one or more embodiments, the user interface 900 may include scheduling of medication administrations to ensure proper medication of the patient while distributing the workload over a period of time to ensure that all medication is given promptly.

In operation, the user interface 900 may be provided by the control system 110, and/or one or more of the healthcare systems 120A-D, for display on a screen, such as a screen of one or more of a user devices 140A being accessed by a healthcare professional, and/or a screen or monitor associated with one or more of the healthcare systems 120A-D. For example, the control system 110 may receive messages from one or more of the healthcare systems 120A-D, and/or one or more of the healthcare devices 130A-F, that relate to in-progress actions and/or future actions that are being performed with the facilitation of the healthcare professional, and/or that are being performed proximally to the healthcare professional, as indicated by the location of the user device 140A. The control system 110 may parse the received messages to obtain the information displayed on the user interface 900, and/or the received messages may be displayed on the user interface 900.

In one or more embodiments, the user interface 900 may automatically be provided to the user device 140A of a healthcare professional when the healthcare professional is located within a proximity of the patient and/or within a proximity of one or more healthcare devices 130A-F that are providing, and/or facilitating with providing, healthcare to one or more patients. Thus, the user device 140A of a healthcare professional may automatically display, and/or prepare for display, information related to in-progress and/or future actions that are being performed, and/or will be performed, proximally to the location of the healthcare professional. Accordingly, the information displayed on the user interface 900 may change as the healthcare professional moves throughout the healthcare facility.

In one or more embodiments, the user device 140A may receive information for displaying the user interface 900 when the healthcare professional is proximally located to in-progress, or future actions, but the user device 140A may not display the user interface 900 until prompted by the healthcare professional. For example, the user device 140A may receive the information for displaying the user interface 900 and may then display a notification to the healthcare professional indicating that the information is available, and requesting whether the healthcare professional would like the user interface 900 to be displayed on the user device 140A and/or on a display or monitor proximally located to the user device 140A.

The information displayed on the user interface 900 may be updated in real-time as the control system 110 and/or one or more of the healthcare systems 120A-D receives messages from the healthcare devices 130A-F, such as while orders are being administered to patients. In one or more embodiments, the user interface 900 may be used to verify the administration of orders, such as a healthcare professional verifying that a medication scheduled for administration and/or a medication being administered coincides with the ordered medication. In one or more embodiments, a healthcare professional may be able to select, such as touch or click on, an order and the user interface 900 may display a picture of the medication being administered, or about to be administered.

FIG. 10 illustrates an example user interface 1000 that may be implemented in a pharmacy workflow management system in accordance with one or more embodiments. Not all of the depicted components may be required, however, and one or more embodiments may include additional components not shown in the figure. Variations in the arrangement and type of the components may be made without departing from the spirit or scope of the claims as set forth herein. Additional, different or fewer components may be provided.

The user interface 1000 may display information relating to patients in a healthcare facility and/or in an area of a healthcare facility, such as alerts or notifications related to healthcare being administered to patients in a healthcare facility. For example, the user interface 1000 may display a graphical representation of each room in an area of the healthcare facility, the name of the patient occupying each room, if any, and any alerts or notifications that apply to any of the displayed patients. In the user interface 1000, an alert or notification is indicated by an asterisk (“*”); however, any other graphical indicator may be used to indicate an alert and/or notification.

In operation, the user interface 1000 may be provided by the control system 110, and/or one or more of the healthcare systems 120A-D, for display on a screen, such as a screen of one or more of the user devices 140A-C, and/or a screen or monitor associated with one or more of the healthcare systems 120A-D. For example, the control system 110 may receive messages from one or more of the healthcare devices 130A-F related to actions being performed by the or more healthcare devices 130A-F. The control system 110 may parse the received messages to determine whether any alerts and/or notifications should be displayed via the user interface 1000, such as whether any discrepancies and/or errors are identified from the received messages.

In one or more embodiments, the user interface 1000 may automatically be provided to the user device 140A of a healthcare professional when the healthcare professional is located within a proximity of the area of the healthcare facility represented on the user interface 1000. Thus, the user device 140A of a healthcare professional may automatically display, and/or prepare for display, the user interface 1000 when a healthcare professional is proximally located to the represented area. Accordingly, the information displayed on the user interface 1000 may change as the healthcare professional moves throughout the healthcare facility.

In one or more embodiments, the user device 140A may receive information for displaying the user interface 1000 when the healthcare professional is located proximally to the represented area, but the user device 140A may not display the user interface 1000 until prompted by the healthcare professional. For example, the user device 140A may receive the information for displaying the user interface 1000 and may then display a notification to the healthcare professional indicating that the information is available, and requesting whether the healthcare professional would like the user interface 1000 to be displayed on the user device 140A and/or on a display or monitor proximally located to the user device 140A.

The information displayed on the user interface 1000 may be updated in real-time as the control system 110 and/or one or more of the healthcare systems 120A-D receives messages from the healthcare devices 130A-F, such as while orders are being administered to patients.

In one or more embodiments, the user interface 1000 may display the status of each patient's infusion, and when an alert occurs, the box representing the patient's room flashes red to attract attention to the alert. Accordingly, a healthcare professional accessing the user interface 1000 may be able to quickly and easily identify the patient from the user interface, such as at a nursing station, and take appropriate action to address the condition causing the alert. In one or more embodiments, certain alerts that have been identified as particularly important events may be displayed on multiple screens throughout the healthcare facility, such as in the pharmacy.

In one or more embodiments, the user interface 1000 may also be used for updating administrative records of the healthcare facility. For example, if a patient changes rooms, a healthcare professional can transmit a notification of the room change to the control system 110 by selecting the patient's name, and dragging that patient to the new room. In addition, the user interface 1000 may be updated to reflect the room change.

FIG. 11 conceptually illustrates electronic system 1100 with which one or more embodiments of the subject technology may be implemented. Electronic system 1100, for example, can be, or can include, the control system 110, the interface system 320, one or more of the healthcare systems 120A-D, one or more of the healthcare devices 130A-F, one or more of the user devices 140A-C, a desktop computer, a laptop computer, a tablet computer, a phone, a personal digital assistant (PDA), and/or generally any electronic device that transmits signals over a network. Such an electronic system includes various types of computer readable media and interfaces for various other types of computer readable media. Electronic system 1100 includes bus 1108, processing unit(s) 1112, system memory 1104, read-only memory (ROM) 1110, permanent storage device 1102, input device interface 1114, output device interface 1106, and network interface 1116, or subsets and variations thereof.

Bus 1108 collectively represents all system, peripheral, and chipset buses that communicatively connect the numerous internal devices of electronic system 1100. In one or more embodiments, bus 1108 communicatively connects processing unit(s) 1112 with ROM 1110, system memory 1104, and permanent storage device 1102. From these various memory units, processing unit(s) 1112 retrieves instructions to execute and data to process in order to execute the processes of the subject disclosure. The processing unit(s) can be a single processor or a multi-core processor in different embodiments.

ROM 1110 stores static data and instructions that are needed by processing unit(s) 1112 and other modules of the electronic system. Permanent storage device 1102, on the other hand, is a read-and-write memory device. This device is a non-volatile memory unit that stores instructions and data even when electronic system 1100 is off. One or more embodiments of the subject disclosure use a mass-storage device (such as a magnetic or optical disk and its corresponding disk drive) as permanent storage device 1102.

Other embodiments use a removable storage device (such as a floppy disk, flash drive, and its corresponding disk drive) as permanent storage device 1102. Like permanent storage device 1102, system memory 1104 is a read-and-write memory device. However, unlike storage device 1102, system memory 1104 is a volatile read-and-write memory, such as random access memory. System memory 1104 stores any of the instructions and data that processing unit(s) 1112 needs at runtime. In one or more embodiments, the processes of the subject disclosure are stored in system memory 1104, permanent storage device 1102, and/or ROM 1110. From these various memory units, processing unit(s) 1112 retrieves instructions to execute and data to process in order to execute the processes of one or more embodiments.

Bus 1108 also connects to input and output device interfaces 1114 and 1106. Input device interface 1114 enables a user to communicate information and select commands to the electronic system. Input devices used with input device interface 1114 include, for example, alphanumeric keyboards and pointing devices (also called “cursor control devices”). Output device interface 1106 enables, for example, the display of images generated by electronic system 1100. Output devices used with output device interface 1106 include, for example, printers and display devices, such as a liquid crystal display (LCD), a light emitting diode (LED) display, an organic light emitting diode (OLED) display, a flexible display, a flat panel display, a solid state display, a projector, or any other device for outputting information.

One or more embodiments may include devices that function as both input and output devices, such as a touchscreen. In these embodiments, feedback provided to the user can be any form of sensory feedback, such as visual feedback, auditory feedback, or tactile feedback; and input from the user can be received in any form, including acoustic, speech, or tactile input.

Finally, as shown in FIG. 11, bus 1108 also couples electronic system 1100 to a network (not shown) through network interface 1116. In this manner, the computer can be a part of a network of computers (such as a local area network (“LAN”), a wide area network (“WAN”), or an Intranet, or a network of networks, such as the Internet. Any or all components of electronic system 1100 can be used in conjunction with the subject disclosure.

Many of the above-described features and applications may be implemented as software processes that are specified as a set of instructions recorded on a computer readable storage medium (alternatively referred to as computer-readable media, machine-readable media, or machine-readable storage media). When these instructions are executed by one or more processing unit(s) (e.g., one or more processors, cores of processors, or other processing units), they cause the processing unit(s) to perform the actions indicated in the instructions. Examples of computer readable media include, but are not limited to, RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, ultra density optical discs, any other optical or magnetic media, and floppy disks. In one or more embodiments, the computer readable media does not include carrier waves and electronic signals passing wirelessly or over wired connections, or any other ephemeral signals. For example, the computer readable media may be entirely restricted to tangible, physical objects that store information in a form that is readable by a computer. In one or more embodiments, the computer readable media is non-transitory computer readable media, computer readable storage media, or non-transitory computer readable storage media.

In one or more embodiments, a computer program product (also known as a program, software, software application, script, or code) can be written in any form of programming language, including compiled or interpreted languages, declarative or procedural languages, and it can be deployed in any form, including as a stand alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

While the above discussion primarily refers to microprocessor or multi-core processors that execute software, one or more embodiments are performed by one or more integrated circuits, such as application specific integrated circuits (ASICs) or field programmable gate arrays (FPGAs). In one or more embodiments, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way) all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more embodiments, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

As used herein, the phrase “at least one of” preceding a series of items, with the term “and” or “or” to separate any of the items, modifies the list as a whole, rather than each member of the list (i.e., each item). The phrase “at least one” of does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

The predicate words “configured to”, “operable to”, and “programmed to” do not imply any particular tangible or intangible modification of a subject, but, rather, are intended to be used interchangeably. In one or more embodiments, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

A phrase such as “an aspect” does not imply that such aspect is essential to the subject technology or that such aspect applies to all configurations of the subject technology. A disclosure relating to an aspect may apply to all configurations, or one or more configurations. An aspect may provide one or more examples of the disclosure. A phrase such as an “aspect” may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure. A phrase such an “embodiment” may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples of the disclosure. A phrase such as a “configuration” may refer to one or more configurations and vice versa.

The word “exemplary” is used herein to mean “serving as an example, instance, or illustration.” Any embodiment described herein as “exemplary” or as an “example” is not necessarily to be construed as preferred or advantageous over other embodiments. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112, sixth paragraph, unless the element is expressly recited using the phrase “means for” or, in the case of a method claim, the element is recited using the phrase “step for.”

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein, but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method for managing pharmacy workflow, the method comprising:
receiving, from at least one healthcare device, at least one information item pertaining to progress of delivery of a first medication of a first order, wherein additional medication is required for the first order when the delivery is complete;
receiving a second order for a second medication from a healthcare system, the second medication being different than the first medication; and
scheduling a first preparation of the additional medication for the first order and a second preparation of the second medication of the second order such that the additional medication is prepared and available upon completion of the delivery of the first medication, wherein the scheduling is based at least in part on the at least one information item.

2. The method of claim 1, wherein the at least one information item indicates that the delivery of the first medication is complete.

3. The method of claim 1, wherein the at least one information item indicates that the delivery of the first medication is not on schedule.

4. The method of claim 1, wherein the at least one information item indicates that a medication was received by the at least one healthcare device.

5. The method of claim 4, further comprising:
verifying, with either optical recognition or bar code technology, that the medication received by the at least one healthcare device corresponds to first medication of the first order.

6. The method of claim 1, further comprising:
providing a report regarding the first preparation of the additional medication and the second preparation of the second medication.

7. The method of claim 6, further comprising:
displaying the report on a screen.

8. The method of claim 1, further comprising:
controlling the scheduling through a module.

9. The method of claim 8, wherein the controlling the scheduling through the module further comprises:
modifying the scheduling using an interface.

10. A non-transitory machine readable medium embodying instructions that, when executed by a machine, cause the machine to perform a method for managing pharmacy workflow, the method comprising:
receiving, from a plurality of healthcare devices, information pertaining to progresses of deliveries of a plurality of first medications of a plurality of first orders, wherein additional medication is required for at least one of the plurality of first orders when the delivery of at least one of the plurality of first medications of the at least one of the plurality of first orders is complete;
receiving a plurality of second orders for a plurality of second medications from a healthcare system; and
determining a schedule for preparing the additional medication for the at least one of the plurality of first orders and the plurality of second medications of the plurality of second orders based at least in part on the progresses of the deliveries of the plurality of first medications.

11. The non-transitory machine readable medium of claim 10, the method further comprising:
transmitting the schedule to at least one user device that includes a display.

12. The non-transitory machine readable medium of claim 11, the method further comprising:
receiving, from a first healthcare device of the plurality of healthcare devices, a first indication that the delivery of a first medication of the plurality of first medications is complete.

13. The non-transitory machine readable medium of claim 12, the method further comprising:
receiving, from a second healthcare device of the plurality of healthcare devices, a second indication that the delivery of a second medication of the plurality of first medications is behind schedule.

14. The non-transitory machine readable medium of claim 13, the method further comprising:
receiving, from a third healthcare device of the plurality of healthcare devices, a third indication that a third medication of a first order of the plurality of first orders was received.

15. The non-transitory machine readable medium of claim 14, wherein the healthcare system comprises a physician order entry system, the first healthcare device of the plurality of healthcare devices comprises an infusion device, the second healthcare device of the plurality of healthcare devices comprises a dispensing device, and the at least one user device comprises at least one of a mobile phone or a tablet device.

16. The non-transitory machine readable medium of claim 14, the method further comprising:
validating, based on either optical recognition or bar code technology, that the third medication received by the third healthcare device corresponds to medication information of the first order of the plurality of first orders.

17. The non-transitory machine readable medium of claim 16, wherein the validating is performed with the optical recognition, and the method further comprising:
transmitting a picture of the third medication to the at least one user device; and
receiving a validation from the at least one user device that the third medication has been optically recognized.

18. The non-transitory machine readable medium of claim 11, the method further comprising:
providing, to the at least one user device, an interface for modifying the schedule;
receiving, from the user device, a modification to the schedule; and
modifying the schedule based at least in part on the received modification.

19. The non-transitory machine readable medium of claim 18, the method further comprising:
receiving, from the plurality of healthcare devices, additional information pertaining to progresses of deliveries of the plurality of first medications of the plurality of first orders; and
modifying the schedule based at least in part on the additional information.

20. The non-transitory machine readable medium of claim 19, wherein the additional information comprises an indication that one of the deliveries of one of the plurality of first medications of one of the plurality of first orders is not on schedule.

21. A system, comprising:
one or more processors; and
a memory including instructions that, when executed by the one or more processors, cause the one or more processors to:
receive a plurality of orders, each order being for administration of one of a plurality of medications to one of a plurality of patients with facilitation from one of a plurality of healthcare devices;
receive a plurality of information items each pertaining to a progress of the administration of the one of the plurality of medications to one of the plurality of patients with facilitation from one of the plurality of healthcare devices; and
provide a report that indicates the progress of the administration of each of the plurality of medications of each of the plurality of orders.

22. The system of claim 21, wherein the instructions, when executed by the one or more processors, further cause the one or more processors to:
determine a schedule for preparing additional orders for additional medications based at least in part on the report.

23. The system of claim 21, wherein the instructions, when executed by the one or more processors, further cause the one or more processors to:
display the report on a screen in a pharmacy.

24. The system of claim 21, wherein the instructions, when executed by the one or more processors, further cause the one or more processors to:
determine, based on the plurality of information items and the plurality of orders, that at least one error exists with respect to the administration of one of the plurality of medications.

25. The system of claim 24, wherein the report further comprises an indication of the at least one error with respect the administration of the one of the plurality of medications.

26. The system of claim 25, wherein the at least one error is based at least in part on an incorrect medication being administered or a drug interaction.

27. The system of claim 21, wherein the plurality of orders are received from a healthcare system and over a communication network, and the plurality of information items are received over the communication network from the plurality of healthcare devices that are facilitating the administration of the plurality of medications.

28. The system of claim 27, wherein the healthcare system comprises at least one of a pharmacy information system, a hospital information system, or a physician order entry system and at least one of the plurality of healthcare devices comprises an infusion device or a smart bed and at least one of the plurality of healthcare devices comprises a patient feeding device, a smart bed, an infusion device, a drug delivery device, a patient monitoring device, or a respiratory device.

29. The system of claim 28, wherein the report is sortable based on a plurality of priorities of the plurality of orders.

30. A method for managing pharmacy workflow, the method comprising:
receiving, from at least one healthcare device, at least one information item pertaining to progress of delivery of a first medication of a first order;
receiving a second order for the first medication from a healthcare system; and
scheduling a preparation for the second order of the first medication such that the second order for the first medication is filled and available upon completion of the delivery of the first order of the first medication.
